(19) United States
(12) Patent Application Publication
(10) Pub. No.: US 2009/0287104 A1
(43) Pub. Date: Nov. 19, 2009

(54) Method and Monitoring Device for Monitoring a Contacting of Electrodes of an ECG Measuring Device

(76) Inventors: Nikolaus Demharter, Dormitz (DE); Michael Frank, Erlangen (DE); Sven Heggen, Erlangen (DE); Jurgen Rossler, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/437,153

(22) Filed: May 7, 2009

(30) Foreign Application Priority Data
May 14, 2008 (DE) 10 2008 023 533.4

(51) Int. Cl. A61B 5/0402

(52) U.S. Cl. 600/509

(57) ABSTRACT

A method for monitoring a contacting of electrodes of an ECG measuring devices is provided. At least three signals each from a first and a second input signal are generated. The first and the second input signal emanate from different electrodes and each electrode supplies one of the input signals to at least two of the signals generated by a first signal processing unit. The three signals are compared with a reference value. Inadequately contacted electrodes are identified based on the comparisons and are displayed. A monitoring device for implementing the method is provided. The monitoring device comprises a first signal processing unit connected to the electrodes at an input side, a second signal processing unit for identifying the inadequately contacted electrodes by receiving and processing at least three signals generated by the first signal processing unit, and a display unit for displaying the inadequately contacted electrodes.

### CROSS REFERENCE TO RELATED APPLICATIONS

This application claims priority of German application No. 10 2008 023 533.4 filed May 14, 2008, which is incorporated by reference herein in its entirety.

### FIELD OF THE INVENTION

The invention relates to a method and a monitoring device for monitoring a contacting of electrodes of an ECG measuring device.

### BACKGROUND OF THE INVENTION

ECG measuring devices are used primarily to measure and monitor the heart function of a patient. For this purpose, typically by means of at least two electrodes the summation voltage of the electrical activity of the heart muscle fibers is measured as a so-called “ECG signal”.

In order to be able to measure such an ECG signal, it is necessary to establish a contact of the electrodes with the examination subject, for example the patient. The electrodes are therefore conventionally attached firmly to the examination subject, for example by adhering them to the chest of a patient.

Even if the contacting of just one electrode with the examination subject has not been adequately established, the quality of the ECG signal deteriorates considerably. Especially if the ECG signal is to be used to trigger the recording of images by a medical diagnostic device such as for example an MRI scanner or a CT scanner, then false triggering will result also in a deterioration of the image quality of these scans. In each case this is to be avoided.

Currently, in order to detect an inadequate contacting of the electrodes, voltages are impressed via a plurality of megohm resistors on the electrodes so that, given contacting with a patient, a low measuring current flows through the patient. As a function of the magnitude of the transfer resistance of the contacting between patient and electrode, the voltage across the electrodes varies. It is then established, for example by means of an analogue voltage monitoring of all of the electrodes, whether there is globally a good contact. In this case, it is checked whether a preset threshold value for the voltage measured across the electrodes has been exceeded. If this is true of even just one electrode, this is signaled as a global “electrode fault” for example by the lighting-up of a small lamp. As in this case it is not known which electrode is inadequately contacted, all of the electrodes have to be exchanged. This is costly in terms of both the time involved and the material outlay because even electrodes that are properly contacted are removed and replaced. If one wished to determine individual inadequately contacted electrodes in this way, it would entail an expensive installation of further cabling, which would increase the susceptibility to faults of the ECG measuring device and in particular impair screening in a magnetic resonance device.

### SUMMARY OF THE INVENTION

The object of the invention is therefore to specify a method and a monitoring device for monitoring a contacting of electrodes of an ECG measuring device, which allow rapid and reliable detection of electrode faults and enable fast and advantageous elimination of these electrode faults.

The object is achieved according to the invention by a method and by a monitoring device as claimed in the claims.

The method according to the invention for monitoring a contacting of electrodes with an examination subject is suitable for ECG measuring devices comprising at least three electrodes and comprises the following steps:
generate at least three signals each from a first and a second input signal, the first and the second input signal of a signal emanating from in each case different electrodes, in such a way that each electrode of the ECG measuring device supplies one of the input signals to at least two of the signals generated by the first signal processing unit,
compare each of the at least three signals with at least one reference value,
identify inadequately contacted electrodes of the ECG measuring device on the basis of the effected comparisons,
display the inadequately contacted electrodes.

A monitoring device according to the invention allows the implementation of a method according to the invention. For this purpose, the monitoring device comprises a first signal processing unit, which at the input side is connected to electrodes of an ECG measuring device, a second signal processing unit, which is designed to identify inadequately contacted electrodes by receiving and processing at least three signals of the first signal processing unit, and a display unit for displaying the electrodes identified as inadequately contacted.

A method according to the invention and a monitoring device according to the invention allow reliable and rapid determination of electrodes that are inadequately contacted with an examination subject. Thus, it may be displayed to a user precisely which electrodes are to be exchanged in order to obtain a high-quality ECG signal. It is therefore possible to save both time as well as money and material because only the electrodes that are inadequately contacted need be exchanged.

### BRIEF DESCRIPTION OF THE DRAWINGS

Further advantages and details of the present invention arise from the following description of exemplary embodiments and from the drawings. The cited examples do not represent a restriction of the invention. The drawings show:

FIG. 1 a schematic representation of a monitoring device according to the invention,

FIG. 2 a schematic sequence diagram of a method according to the invention.

### DETAILED DESCRIPTION OF THE INVENTION

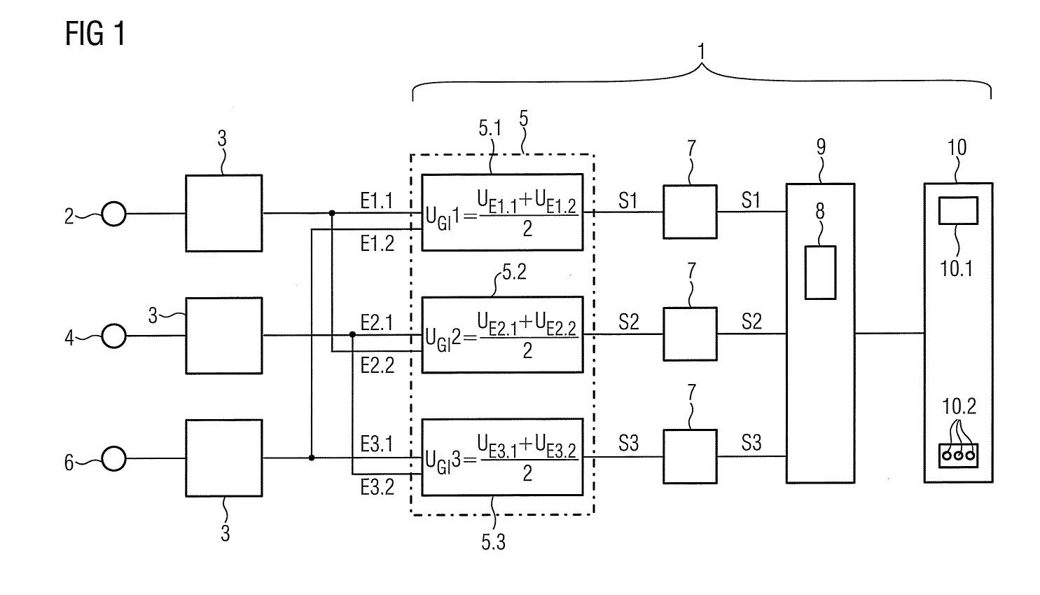

FIG. 1 shows schematically a monitoring device 1 according to the invention. The monitoring device 1 comprises a first signal processing unit 5, which in the present embodiment is connected at the input side to three electrodes 2, 4, 6 of an ECG measuring device. Without this being intended to represent a restriction, all of the explanations below refer to an ECG measuring device having three electrodes.

Filter/amplifier units 3 are optionally disposed in the signal-carrying connection lines between the electrodes 2, 4, 6 and the first signal processing unit 5 in order to improve signal transmission. Such filter/amplifier units 3 are known and regularly used in conjunction with ECG electrodes. For the sake of clarity, further parts of the ECG measuring device are not represented explicitly, especially as these parts are known from the background art. The electrodes 2, 4, 6 of the ECG measuring device are attached to a non-illustrated examination subject in order to measure ECG signals.

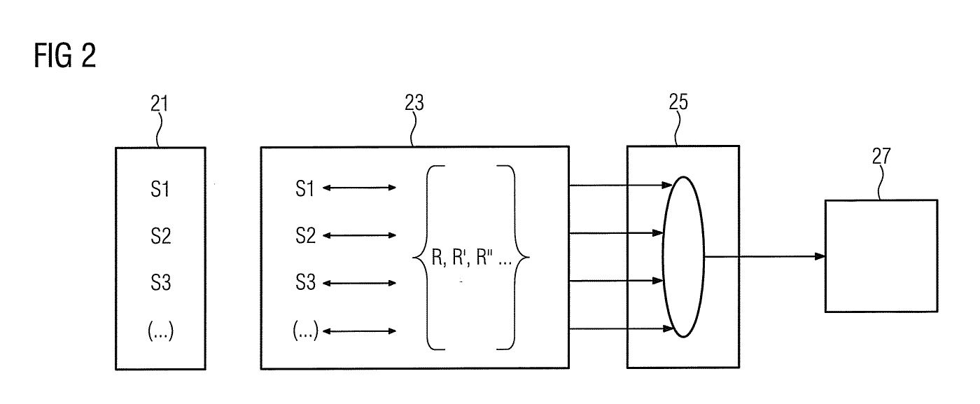

As represented in FIG. 1, the first signal processing unit 5 receives from each electrode 2, 4, 6 input signals E1.1, E1.2, E2.1, E2.2, E3.1, E3.2 and processes these into signals S1, S2, S3. In FIG. 2, in which the sequence of a method according to the invention is schematically represented, the generation of the signals S1, S2, S3 is represented by step 21.

Here, to generate each signal S1, S2, S3 in each case a first and a second input signal Ea.1 and Ea.2 (a=1, 2, 3 for each signal S1, S2, S3) are used, with in each case the first and the second input signal Ea.1 and Ea.2 emanating from different electrodes 2, 4, 6. Each electrode 2, 4, 6 in this case supplies one of the input signals E1.1, E1.2, E2.1, E2.2, E3.1, E3.2 to at least two of the generated signals S1, S2, S3.

In this case, the input signals E1.1, E1.2, E2.1, E2.2, E3.1, E3.2 are to be selected in such a way that an inadequate contacting of an electrode 2, 4, 6 manifests itself in the input signals emanating from this electrode and hence also in the corresponding signals. For example, an inadequate contacting of electrode 2 is to manifest itself in the input signals El. 1 and E2.2 and hence in the signals S1 and S2.

The first signal processing unit 5 of the monitoring device 1 is advantageously a common-mode measuring unit that determines the common-mode component of the pairs of input signals Ea.1 and Ea.2 described above.

This occurs for example, in the manner represented in the exemplary embodiment of FIG. 1, by means of common-mode measuring devices 5.1, 5.2, 5.3, which determine from each two input signals Ea.1 and Ea.2 as signals S1, S2, S3 the common-mode component

$${U}_{G 1} a = \frac{{U}_{Ea . 1} + {U}_{Ea . 2}}{2} ( a = 1 , 2 , 3 ) .$$

In the event of an inadequate contacting of an electrode, the transfer resistance between the electrode and the examination subject is increased. Thus, the inadequate contacting will manifest itself also in an increase of a common-mode voltage measured by such an inadequately contacted electrode.

Here, it is particularly advantageous to obtain the common-mode components directly from an ECG signal processing unit that the ECG measuring device already comprises, for example from a differential amplifier unit. In such an embodiment of the monitoring device 1 the first signal processing unit 5 is therefore part of an existing ECG signal processing unit of the ECG measuring device for measuring the ECG signals. It is therefore possible to dispense with a separate first signal processing unit 5. Instead, the ECG measuring device may be used synergetically to monitor the contacting of its electrodes in that the signals S1, S2, S3 are obtained from the ECG measurement signals without any further units being inserted therebetween.

From the first signal processing unit 5 the signals S1, S2, S3 are transmitted to a second signal processing unit 9. In the present case, the signals S1, S2, S3 are advantageously digitized by means of digitizing units 7 that are disposed in the corresponding signal paths.

The second signal processing unit 9 at the input side receives the signals S1, S2, S3 and compares them individually with at least one reference value R, R′, R″ (step 23 in FIG. 2). On the basis of results of the effected comparisons, the second signal processing unit 9 by combination identifies inadequately contacted electrodes (step 25 in FIG. 2).

In this case, in step 23 for example a respective value of each of the signals S1, S2, S3 is compared with at least one preset reference value R, R′, R″ and it is decided whether the value of the signal S1, S2, S3 is greater or smaller than the reference value R, R′, R″. Thus, the signals S1, S2, S3 are compared in accordance with orders of magnitude of their values. The at least one reference value R, R′, R″ therefore functions as a threshold value for classifying the signals S1, S2, S3 in accordance with an order of magnitude of their values.

In step 25 the results of these comparisons are gated by means of logic operations in order to identify inadequately contacted electrodes. In so doing, in each case it is taken into consideration how an inadequate contacting of an electrode manifests itself in the signals S1, S2, S3.

For example, in the previously described situation where the signals S1, S2, S3 are determined as common-mode signals of the voltages across the electrodes 2, 4, 6, what is being searched for is an electrode fault, i.e. an inadequate contacting in the case of the electrode, for which both common-mode signals S1, S2, S3 that were generated from input signals of this electrode have an increased value.

By way of further explanation, the following table presents several measurement examples of measured common-mode signals S1, S2, S3. Entered in the first column of this table is the measured value of the signal S1, in the second column the measured value of the signal S2, in the third column the measured value of the signal S3 and in the fourth column the electrode identified from this as inadequately contacted:

| S1 [mV] | S2 [mV] | S3 [mV] | Faulty electrode |
|---|---|---|---|
| 31 | 30 | 32 | — |
| 127 | 805 | 24 | 2 |
| 797 | 23 | 125 | 6 |
| 25 | 124 | 798 | 4 |
| 1077 | 802 | 117 | 2, 6 |
| 125 | 1075 | 795 | 2, 4 |
| 794 | 117 | 1075 | 4, 6 |
| 1094 | 1094 | 1089 | 2, 4, 6 |

A possible choice of the thresholds usable for these measurement examples is for example: R=100 mV, R′=750 mV and R″=1000 mV.

In a simple manner the respective thresholds may be determined for example by adjusting a transfer resistance for each electrode by means of a phantom and measuring the associated common-mode voltage.

By means of logic operations it is then possible to identify inadequately contacted electrodes. Possible logic operations are for example:

IF S1>R AND S2>R′ THEN electrode 2

IF S1>R′ AND S3>R THEN electrode 6

IF S2>R AND S3>R′ THEN electrode 4

IF S1>R″ AND (S2<R″ OR S3 <R″) THEN electrodes 2 and 6

IF S2>R″ AND (S1<R″ OR S3 <R″) THEN electrodes 2 and 4

IF S3>R″ AND (S1<R″ OR S2<R″) THEN electrodes 4 and 6

IF S1>R″ AND S2>R″ AND S3>R″ THEN electrodes 2 and 6.

In order that signals S1, S2, S3 of inadequately contacted electrodes reach a defined value, a potential lying across the examination subject may be adjusted by means of a further electrode (not shown) to a defined value, for example by grounding to a value of 0 V. By means of such an adjustment of a defined potential at the examination subject a reference value R, R′ for comparing with the signals S1, S2, S3 may easily be preset, which indicates an inadequate contacting.

In an advantageous exemplary embodiment the second signal processing unit 9 takes the form of a DSP unit (DSP: digital signal processing) and comprises a logic/memory unit 8 for executing logic operations and for storing for example comparison results and reference values. With such a second signal processing unit 9 at least one of the steps, compare 23 and identify 25, may be effected digitally.

A result of the identification of inadequately contacted electrodes is communicated in a conventional manner from the second signal processing unit 9 to a display unit 10. The display unit 10 displays for a user electrodes that have been identified as inadequately contacted (step 27 in FIG. 2). For this purpose, the display unit 10 comprises for example a display 10.1, at which information at least about the identity of the electrodes identified as inadequately contacted is displayed for a user. Additionally or alternatively the display unit 10 may comprise at least one means of illumination 10.2 per electrode 2, 4, 6, for example in the form of LEDs. In this case, an inadequate contacting may be displayed for the user for example by virtue of a means of illumination 10.2 associated with an electrode 2, 4, 6 lighting up or not lighting up. An alternative possibility is a coding of the functionality of the relevant electrode by means of a specific color, for example “red” for “fault” and “green” for “operational”, of a light emitted by the respective means of illumination 10.2.

1.-13. (canceled)

14. A method for monitoring an electrode contacting of an ECG measuring device comprising at least three electrodes with an examination subject, comprising:
emanating input signals each comprising a first input signal and a second input signal from different electrodes of the at least three electrodes;
generating at least three signals each from the first input signal and the second input signal, wherein each of the at least three electrodes supplies one of the first input signal and the second signal to at least two of the generated signals;
comparing the at least three signals with a reference value;
identify an inadequately contacted electrode based on the comparison; and
displaying the inadequately contacted electrode.

15. The method as claimed in claim 14, wherein the identification comprises a logic operation.

16. The method as claimed in claim 14, wherein the at least three signals are obtained from ECG measurement signals.

17. The method as claimed in claim 14, wherein the reference value is preset by a potential generated at the examination subject.

18. The method as claimed in claim 14, wherein the at least three signals are compared in accordance with magnitude order of values.

19. The method as claimed in claim 14, wherein the comparison or the identification is effected digitally.

20. A monitoring device for monitoring an electrode contacting of an ECG measuring device comprising at least three electrodes with an examination subject, comprising:
a first signal processing unit connected to the electrodes that generates at least three signals each from a first input signal and a second input signal emanating from different electrodes of the at least three electrodes;
a second signal processing unit connected to the first signal processing unit that processes the at least three signals and identifies an inadequately contacted electrode; and
a display unit that displays the inadequately contacted electrode.

21. The monitoring device as claimed in claim 20, wherein the ECG measuring device comprises an ECG signal processing unit and the first signal processing unit is part of the ECG signal processing unit.

22. The monitoring device as claimed in claim 20, wherein the first signal processing unit is a common-mode measuring unit.

23. The monitoring device as claimed in claim 20, further comprising a digitizing unit disposed in a signal path upstream of the second signal processing unit for converting the at least three signals into digital signals.

24. The monitoring device as claimed in claim 20, wherein the display unit comprises a device for illuminating the electrodes.

25. The monitoring device as claimed in claim 20, wherein the second signal processing unit compares the at least three signals with a reference value.
